(19) United States
(12) Patent Application Publication
(10) Pub. No.: US 2014/0180144 A1
(43) Pub. Date: Jun. 26, 2014

(54) OSCILLOMETRIC NON-INVASIVE BLOOD PRESSURE MEASUREMENTS IN PATIENTS EXPERIENCING ABNORMAL HEARTBEATS

(71) Applicants: Yu Chen, Andover, MA (US); Mohamad M. El-Ghouch, Nashua, NH (US); Zhe Zhang, Westford, MA (US)

(72) Inventors: Yu Chen, Andover, MA (US); Mohamad M. El-Ghouch, Nashua, NH (US); Zhe Zhang, Westford, MA (US)

(73) Assignee: DRAEGER MEDICAL SYSTEMS, INC., Andover, MA (US)

(21) Appl. No.: 14/114,165

(22) PCT Filed: Apr. 26, 2012
(86) PCT No.: PCT/US12/35282

§ 371 Date: Jan. 6, 2014

Related U.S. Application Data
(60) Provisional application No. 61/479,999, filed on Apr. 28, 2011.

(51) Int. Cl. A61B 5/022; A61B 5/0472; A61B 5/021; A61B 5/046; A61B 5/02; A61B 5/0464; A61B 5/0408; A61B 5/04

(52) U.S. Cl. CPC A61B 5/02225; A61B 5/0408; A61B 5/0472; A61B 5/04012; A61B 5/046; A61B 5/02028; A61B 5/0464; A61B 5/02116
USPC 600/494

(57) ABSTRACT

Disclosed is a method for implementation by one or more data processors including acquiring noninvasive blood pressure data using an inflatable cuff applied around a portion of a patient by inflating the cuff to a pressure above the patient's systolic blood pressure, and detecting a first oscillation pulse within the cuff during a deflation step. The method also includes acquiring electrocardiogram (ECG) data from the patient by detecting an ECG waveform corresponding to a first heartbeat using an ECG lead coupled to the patient, classifying the ECG waveform as normal or not normal, aligning, the first oscillation pulse to the ECG waveform corresponding to the first heartbeat if the ECG waveform is classified normal or rejecting the first oscillation pulse if the ECG waveform is classified not normal, and calculating the blood pressure using the aligned first oscillation pulse. Related apparatus, systems, methods and/or articles are described.

### RELATED APPLICATION

The present patent application claims priority under 35 U.S.C. §119(e) to U.S. Provisional Patent Application Ser. No. 61/479,999, filed Apr. 28, 2011, and entitled “Method for Improve Accuracy of Non-Invasive Blood Pressure Measurements in Arrhythmia Patients Using ECG Beat Class Information and System Timing Between NIBP and ECG,” the disclosure of which is incorporated by reference herein in its entirety.

### TECHNICAL FIELD

The subject matter described herein relates generally to the field of medical devices, and more particularly to devices, systems, articles, and methods used to improve the accuracy of non-invasive blood pressure measurements.

### BACKGROUND

Automated blood pressure monitors are commonly used in a variety of medical settings including emergency rooms, intensive and crucial care units, operating rooms and other conventional parts of the patient environment to monitor non-invasive blood pressure (NIBP). One form of NIBP measurement is the auscultatory method, which involves using a sphygmomanometer and stethoscope. An inflatable cuff is positioned around the upper arm of a patient roughly level with the patient's heart. The cuff, attached to a manometer, is inflated until the brachial artery at the elbow is completely occluded. The stethoscope is used to listen to the brachial artery as the pressure in the cuff is slowly released. When blood again starts to flow in the artery, the turbulent flow acting against the arterial walls vibrates creating a whooshing noise known as Korotkoff sounds. The first of five phases of Korotkoff sounds appears at the point when the pressure within the cuff is equivalent to the systolic or peak pressure within the blood vessel. The pressure in the cuff is then further released until laminar blood flow is restored and no sound can be heard which represents the fifth Korotkoff phase, indicative of the diastolic arterial pressure. Because the primary embodiment of the auscultatory method involves a stethoscope and manual interpretation of the Korotkoff sounds by a clinician, it is best suited for periodic as opposed to continuous NIBP readings.

The oscillometric method is another NIBP measurement method that involves the electronic observation of oscillations in the sphygmomanometer cuff pressure caused by the changes in arterial flow resulting from inflating and deflating the cuff. The cuff pressure oscillations are observed using a pressure sensor or transducer and electronics to automatically interpret the oscillations. The inflatable cuff suitably located on the limb of a patient is inflated to a predetermined pressure above the patient's estimated systolic pressure. The cuff pressure is then gradually reduced over a relatively short period of time in predetermined decrements to below diastolic pressure. At each level, the oscillations in the cuff are monitored by the transducer. When blood flow is obstructed and when blood flow is unimpeded, the cuff pressure will be relatively constant and no oscillations present. When some blood flow is present, but restricted, the cuff pressure monitored by the pressure transducer will vary with the cyclic expansion and contraction of the brachial artery generating oscillation signals. As the decrementing continues, the peak amplitudes of the oscillations will normally increase from a lower level to a relative maximum, and thereafter will decrease. These amplitudes form an oscillometric envelope for the patient. The cuff pressure at which the oscillations have a maximum value has been found to be representative of the mean arterial pressure (MAP).

The oscillometric method provides certain advantages in that readings can be taken on a nearly continuous basis with minimal to no risk to the patient as compared to the invasive method. The oscillometric method can also be performed automatically with minimal effort as compared to the auscultatory method, and unlike the auscultatory method, which can directly obtain only systolic and diastolic measurements, can obtain systolic, diastolic and mean arterial pressure measurements. Further, the readings can be performed by a lay person, and because they are automatic, in hospital settings serve as a surveillance tool to monitor blood pressure continuously, providing a trend of any changes in blood pressure and/or alerting a clinician of any significant change.

One of the limitations of oscillometric NIBP monitoring is the ability to reliably classify detected pulses as normal beats of the heart as opposed to abnormal pulses or non-physiological artifacts. Invasive blood pressure monitoring provides a wave form such that the clinician can assess whether or not the reading is affected by an abnormal pulse. Oscillometric NIBP, in contrast, is performed “blind” as there is no reference wave form, just a number.

Oscillation pulses in normal patients will generally have the same shape and can be reliably fit to a curve profile allowing for the calculation of highly accurate blood pressure values for the patient over time. In a patient experiencing abnormal heartbeats, in contrast, oscillation pulses will be affected and can subsequently degrade the accuracy of the blood pressure measurements calculated from the curve profile. Thus, the success of NIBP measurement in patients experiencing abnormal heartbeats and, in particular arrhythmia, remains a significant problem.

### SUMMARY

In one aspect, disclosed is a method for implementation by one or more data processors. The method includes acquiring, by at least one data processor, noninvasive blood pressure data using an inflatable cuff applied around a portion of a patient by inflating the cuff to a pressure above the patient's systolic blood pressure, and detecting a first oscillation pulse within the cuff during a deflation step. The method also includes acquiring, by at least one data processor, electrocardiogram (ECG) data from the patient by detecting an ECG waveform corresponding to a first heartbeat using an ECG lead coupled to the patient. The method also includes classifying, by at least one data processor, the ECG waveform as normal or not normal. The method includes aligning, by at least one data processor, the first oscillation pulse to the ECG waveform corresponding to the first heartbeat if the ECG waveform is classified normal or rejecting the first oscillation pulse if the ECG waveform is classified not normal. The method includes calculating, by at least one data processor, the blood pressure using the aligned first oscillation pulse.

The method can further include detecting, by at least one data processor, a second oscillation pulse within the cuff during a deflation step. The second oscillation pulse can neighbor the first oscillation pulse. The method can further include acquiring, by at least one data processor, electrocardiogram (ECG) data from the patient by detecting a second ECG waveform characterizing a second heartbeat using the ECG lead coupled to the patient. The method can further include classifying, by at least one data processor, the second ECG waveform as normal or not normal. The method can further include aligning, by at least one data processor, the second oscillation pulse to the second ECG waveform corresponding to the second heartbeat if the second ECG waveform is classified normal or rejecting the second oscillation pulse if the second ECG waveform is classified not normal. The method can further include performing, by at least one data processor, oscillation pulse matching between the aligned first oscillation pulse and the aligned second oscillation pulse to identify whether the first oscillation pulse qualifies for inclusion in calculating the blood pressure calculation.

The second oscillation pulse can occur within the same deflation step as the first oscillation pulse. The second oscillation pulse can occur within a different deflation step as the first oscillation pulse. Pulse matching can include identifying similarity between at least one of area, rise time, and pulse amplitude of the first and second oscillation pulses. The first oscillation pulse qualified for inclusion in calculating the blood pressure calculation can be stored to an oscillation pulse profile. The oscillation pulse profile can include additional qualified oscillation pulses detected within the cuff. The method can further include assessing, by at least one data processor, whether the oscillation pulse profile is complete. The method can further include fitting, by at least one data processor, the completed oscillation pulse profile to a curve. Calculating the blood pressure can include calculating systolic, diastolic and mean arterial blood pressure of the patient from the oscillation pulse profile fit to the curve. Calculating the blood pressure can include calculating systolic, diastolic and mean arterial blood pressures of the patient.

The ECG waveform classified as not normal can include premature ventricular contraction (PVC), premature atrial contractions (PAC), atrial fibrillation, atrial flutter, paroxysmal supraventricular tachycardia (PSVT), accessory pathway tachycardias, AV nodal reentrant tachycardia, ventricular tachycardia (V-tach), ventricular fibrillation, long QT syndrome, bradyarrhythmias, sinus node dysfunction, and heart block.

In an interrelated aspect, disclosed is a system including a noninvasive blood pressure monitor to provide noninvasive blood pressure data, an electrocardiogram sensor to provide electrocardiogram data, at least one data processor, memory, coupled to the at least one data processor, storing instructions, which when executed, cause the at least one data processor to perform a method as described above.

Articles of manufacture are also described that comprise computer executable instructions permanently stored on non-transitory computer readable media, which, when executed by a computer, causes the computer to perform operations herein. Similarly, computer systems are also described that may include a processor and a memory coupled to the processor. The memory may temporarily or permanently store (e.g., non-transitorily store, etc.) one or more programs that cause the processor to perform one or more of the operations described herein. In addition, methods described herein can be implemented by one or more data processors either within a single computing system or distributed among two or more computing systems.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a schematic representation of an oscillometric blood pressure monitor system according to an implementation;

FIG. 2 is a raw signal of an inflation and step deflation curve;

FIG. 3 is a blood pressure curve (BP) and a simultaneously acquired ECG over time;

FIG. 4 is a high-level process flow diagram illustrating the data flow and signal processing used to calculate the systolic, diastolic and mean arterial pressure (MAP) for a patient experiencing abnormal heartbeats.

Like reference symbols in the various drawings indicate like elements.

### DETAILED DESCRIPTION

Conventional non-invasive blood pressure (NIBP) monitoring is limited by the monitor's ability to reliably align detected oscillation pulses with normal heartbeats. Normal heart patients (e.g. patients not experiencing arrhythmia or abnormal pulses) produce pressure oscillation pulses that are generally identical to one another that can be saved to a profile and fit to a curve providing highly accurate blood pressure measurements. Patients with abnormal heartbeats, in contrast, tend to present altered pressure oscillation profile shapes such that the accuracy in blood pressure measurements when fit to normal Gaussian curves is degraded.

Described herein are NIBP monitor systems, devices, articles, and methods for use with patients experiencing abnormal heartbeats. The systems, devices, articles, and methods use sensed ECG data to filter out abnormal pressure oscillation pulses from an NIBP detected pulse. An electrocardiogram (ECG or EKG) algorithm has a high reliability in classifying each heartbeat as normal or abnormal. Typically, an ECG has deflections arbitrarily named “P”, “Q,” “R,” “S,” “T,” and sometimes “U.” The deflections and combinations of deflections such as the QRS complex can be used to assess occurrence of irregular heartbeats and arrhythmias.

As will be described in more detail below, each NIBP detected oscillation pulse can be matched or aligned with a corresponding ECG detected heartbeat. If the ECG waveform for the corresponding detected heartbeat is normal, the detected NIBP oscillation pulse aligned to or matched up with that heartbeat will be saved to a database and become part of the oscillation pulse profile. If the ECG waveform for the corresponding detected heartbeat is abnormal, the detected NIBP oscillation pulse aligned to or matched up with that heartbeat will be rejected. The rejected pulse will not be included in the database and will not become part of the oscillation pulse profile. The subject matter described herein allows for the real-time detection and removal of abnormal pulses from the NIBP database using the ECG beat classification information resulting in improved accuracy of NIBP monitor systems in patients experiencing abnormal heartbeats.

The subject matter described herein is appropriate for continuous, automatic NIBP readings and as such can be used for real-time patient monitoring in a variety of medical facilities such as in the hospital ward, operating room, intensive care unit, recovery, the emergency room or another emergency service environments. It should be appreciated that the systems, devices, articles, and methods described herein can be used wherever a patient is being treated and should not be limited to a particular medical facility.

Monitor System

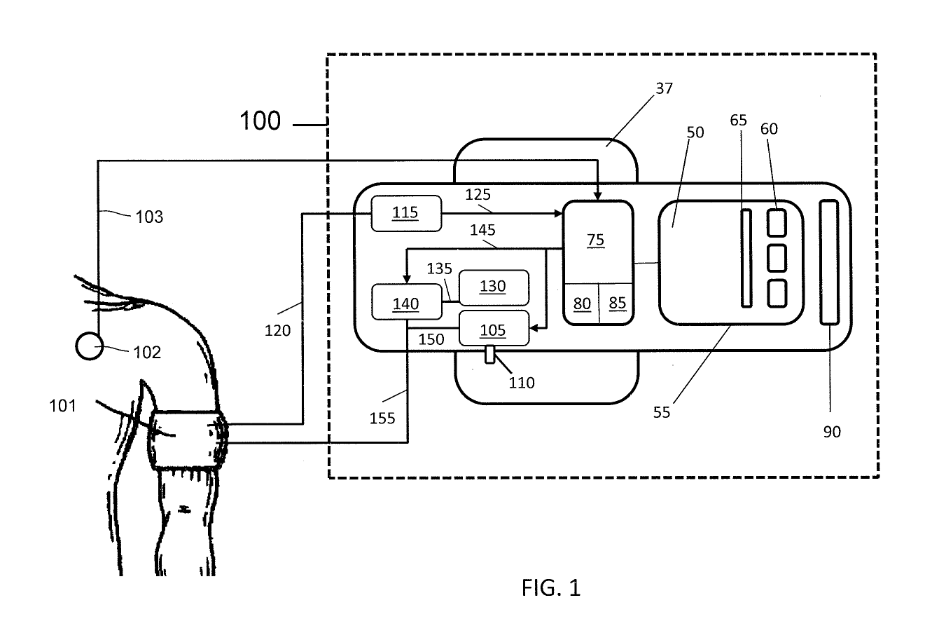

FIG. 1 is a schematic representation of an oscillometric blood pressure monitor system according to an implementation. The monitor system 100 includes a signal acquisition circuit to acquire a pressure signal and generate an oscillometric signal from the pressure signal. The monitor system 100 can be coupled to a cuff 101 to be positioned appropriately on a patient's arm. The cuff 101 can be a conventional flexible inflatable and deflatable cuff 101 that when fully inflated can occlude the brachial artery. The cuff 101 can be deflated using a deflate valve 105 having an exhaust 110 to relieve gradually the arterial occlusion. The deflation of the cuff 101 can occur via a deflate valve 105 controlled by a microprocessor 75.

A pressure transducer 115 can be coupled by a duct 120 to the cuff 101 and used to sense pressure within the cuff 101. As described above, pressure oscillations in the brachial artery can be sensed by changes in the counter-pressure of the cuff 101. These pressure oscillations can be converted into an electrical signal (oscillometric signal) by the pressure transducer 115 and coupled over path 125 to microprocessor 75 for processing. The microprocessor 75 can process the signals from the pressure transducer 115 to produce blood pressure data.

Additionally, a source of pressurized air 130 can be connected via a duct 135 through an inflate valve 140 and a duct 155 coupled to the pressure cuff 101. The inflate valve 140 can be electrically controlled through a connection 145 from the microprocessor 75. The deflate valve 105 can be connected by duct 150 with the duct 155 leading to the cuff 101.

The monitor system 100 can also be used to acquire a signal from ECG electrodes 102 positioned on the patient's body and coupled to the monitor system 100, such as by an ECG lead set 103. The ECG electrodes 102 can be positioned on a patient's body as is known in the art according to a 3-lead, 5-lead, 6-lead, or a 12-lead ECG configuration. The ECG lead set 103 closest to the patient can connect to each electrode 102 or can be integrated into the distal end of the ECG lead set 103 and receive biopotential signals from the patient's body. The other end of the ECG lead set 103 can connect such as via the ECG input connector to the monitor system 100 supplying it with the biopotential signals received from the patient's body. As will be described in more detail below, the monitor system 100 can use information sensed by the ECG regarding beat classification to limit or prevent cuff pressure oscillations resulting from an abnormal heartbeat being used to calculate blood pressure data. It should be appreciated that other sensor systems can be incorporated that provide information regarding the heartbeat, such as a pulse oximeter, or some other type of sensor.

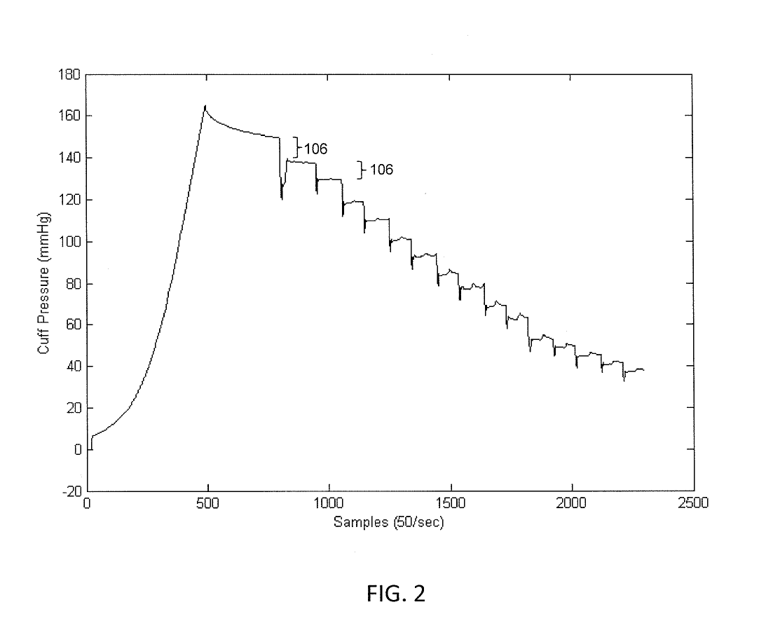

During operation of the system 100 when it is desired to initiate a determination of blood pressure, the microprocessor 75 can provide a signal to open the inflate valve 140 and the deflate valve 105 is closed. Air from the source 130 can be communicated through the inflate valve 140 and duct 155 to inflate the cuff 101 to a desired level. The level is generally above the estimated systolic pressure of the patient. When the pressure in the cuff 101 reaches the predetermined value above the estimated systolic pressure of the patient, the pressure transducer 115 sends a signal on path 125 to the microprocessor 75 indicative of the instantaneous pressure in the cuff 101 to interrupt the inflation of the cuff 101. The signal instructs the inflate valve 140 to close and the step deflation routine is commenced such that the blood pressure measurement can be obtained over a series of deflation steps 106 (see FIG. 2). There is no flow distal to the cuff 101 when it is inflated above systolic pressure, and an oscillation pulse (reflected as a change in the impedance waveform) will appear when the cuff 101 is deflated below systolic pressure. The size of each deflation step can vary, for example, including but not limited to 1, 2, 3, 4, 5, 6, 7, 8 or more mm Hg per deflation step 106. The length of each deflation step can also vary, for example, including but not limited to 1, 2, 3, 4, 5, 6, 7, 8 or more seconds per deflation step 106. Upon completion of each measurement cycle, the deflate valve 105 can be re-opened long enough to relax the cuff pressure substantially completely via the exhaust 110. The deflate valve 105 can remain closed ready for the start of a new measurement cycle.

Again with respect to FIG. 1, the microprocessor 75 can include at least one memory 80 coupled to the microprocessor 75 and including at least one program stored thereon. The memory 80 can be any type of memory 80 capable of storing data and communicating that data to one or more other components of the system 100, such as the processor 75. The memory 80 is in communication with the signal acquisition circuit and can store the cuff pressure signals, the oscillometric signals, and the ECG signals.

The monitor system 100 can include at least one display 50 including a graphical user interface (GUI) 55. The display 50 can provide information to the user such as patient-specific information as well as data being acquired from the patient by the system 100. The display 50 can vary including LCD, LED, plasma, OLED, and the like. The display 50 can be an interactive or touch-sensitive screen having an input device such as a touch screen, a capacitance screen, a resistive screen or the like. The user interface system 55 can include one or more inputs 60 such as fixed buttons associated with fixed functions or changeable functions such as soft keys associated with the display 50. The soft keys can provide functions wherein the function is displayed and the display 50 can change providing different functions in different situations. The fixed input keys can also have a function that changes depending upon the display provided. The inputs 60 can be used, for example, to manually enter values into the device. The measurements can also be automatically provided from either another a parameter measurement obtained on the same device, such as an invasive intra-arterial value, or via another device which is wired or wirelessly connected, such as a manual sphygmomanometer or ECG or other device. The user interface system 55 can also include one or more indicators and/or alarms 65 that may be visual, auditory through a speaker, tactile, and the like.

The system 100 can include a power system 85. The power system 85 can include a connection to an AC wall power through a power cord. The power system 85 can also include internal battery such as a non-rechargeable or a rechargeable battery. Some embodiments may use a rechargeable battery such as a NiCad battery, LiPo battery, NiMH battery or the like.

The monitor system 100 can be a stationary, portable or telemetry-enabled device. For example, the monitor system 100 can be incorporated into a patient monitor including the Infinity® series of patient monitors including Delta, Delta II, Delta XL, or M540 portable patient monitor (Dräger Medical GmbH). The monitor system 100 can include a device that is assigned to a particular patient and located at that patient's bedside. The patient-assigned monitor system 100 can be programmed once for the specific patient to whom the monitor system 100 is assigned. The system 100 can dock with a hardwired docking station 37 located at a patient's bedside and collect data from one or more data acquisition devices besides the NIBP that are additionally acquiring clinical data from a patient.

As mentioned above, the monitor system 100 can include a communication module 90 and can communicate with other devices. The communication can be wired or wireless communication capability for the remote sending and receiving of data, such as via WLAN. The communication module 90 can include a transmitter and/or receiver, IEEE 802.11 (WiFi) connection, ZigBee, RFID, infrared, Bluetooth communication device or the like. The system 100 can be in communication via a network of a hospital or other healthcare-providing entity with a hospital information system (HIS). One or more components of the monitoring system 100 can also be in communication with a central patient monitor.

Pulse Alignment and Matching

For normal patients (i.e. patients not experiencing abnormal heartbeats), the monitoring system 100 can detect and record the amplitude of one or more NIBP oscillation pulses for each cuff pressure level. The pressure transducer 115 can measure the internal cuff pressure and provide an analog signal characterizing the blood pressure oscillatory complexes. As the cuff pressure decreases, the system 100 can detect pressure oscillations and record the pressure oscillation for each cuff pressure in a database or memory 80. An algorithm can save the oscillation pulses after each signal fluctuation is measured and compare the oscillation pulse to a threshold or to a neighboring oscillation pulse. The pulse matching process can read the pulse database and determine whether each pulse is a valid one by comparing a particular oscillation pulse to a neighboring oscillation pulse on the same step or a previous step. The oscillation pulses can become larger to a maximum and then smaller as the cuff pressure continues towards full deflation forming a curve, such as a bell-shaped or Gaussian curve. For normal patients, the matched oscillation pulses will generally be nearly identical in size, shape, and amplitude. As such, when the oscillation pulses are saved to a oscillation pulse profile and fit to a standard curve, the calculation by the processor 75 of the MAP, systolic and diastolic pressures are very accurate. Calculation of blood pressure of patients experiencing abnormal heartbeats using this method, in contrast, can result in inaccuracies.

As will be described in more detail below, the monitoring system 100 can obtain an NIBP waveform including a series of oscillation pulses that can be aligned with corresponding heartbeats shown in an ECG waveform. The monitoring system 100 uses ECG beat classification information to reject and remove abnormal pulses thereby improving the accuracy of the calculated blood pressure.

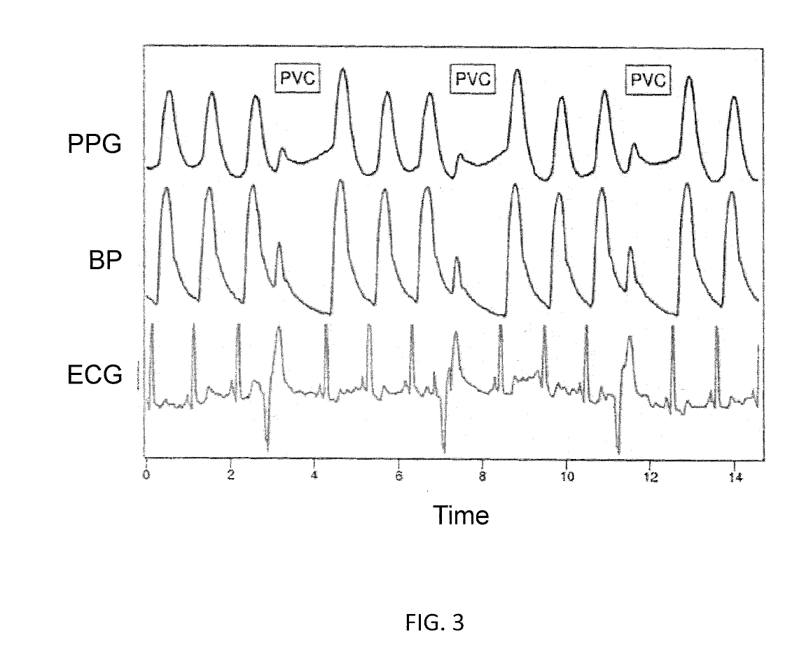

FIG. 3 shows a blood pressure curve (BP) and a simultaneously-acquired ECG tracing of the cardiac cycle (heartbeat), and photoplethysmogram (PPG) showing multiple ECG, BP and PPG waveforms over time. In this example, premature ventricular complex (PVC) pulses are detected. The NIBP pulse occurring at the time of the PVC or another abnormal pulse, if saved in the NIBP pulse profile, would ultimately affect the accuracy of the calculated blood pressure measurement. It should be appreciated that abnormal pulses of the heart can include, but are not limited to, PVC, premature atrial contractions (PAC), atrial fibrillation, atrial flutter, paroxysmal supraventricular tachycardia (PSVT), accessory pathway tachycardias, AV nodal reentrant tachycardia, ventricular tachycardia (V-tach), ventricular fibrillation, long QT syndrome, bradyarrhythmias, sinus node dysfunction, heart block, and others.

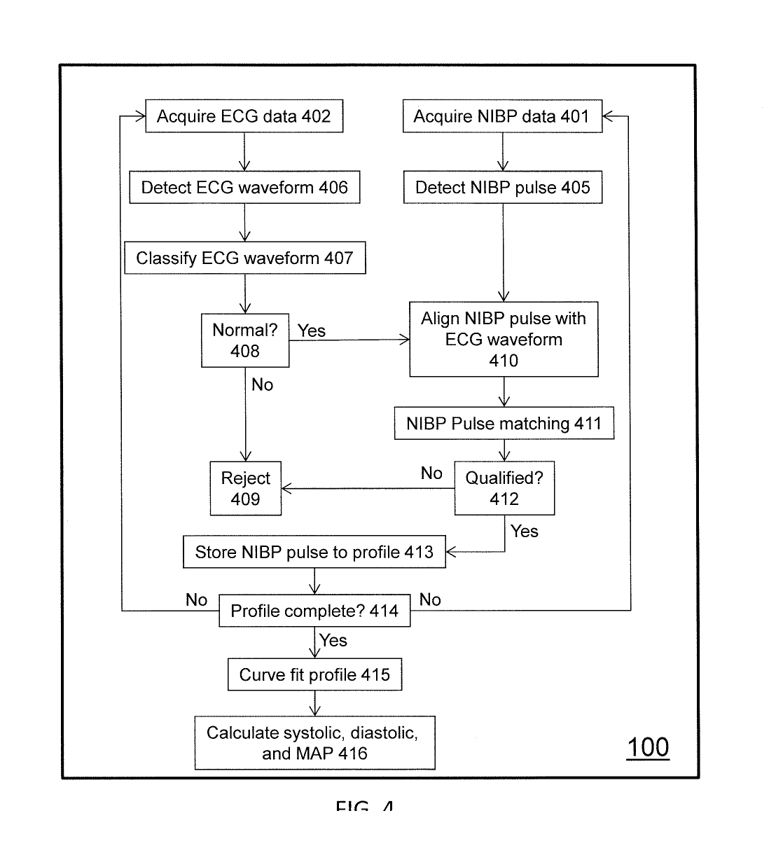

FIG. 4 is a high-level process flow diagram illustrating the data flow and signal processing that can be used to calculate the systolic, diastolic and mean arterial pressure (MAP) for a patient experiencing abnormal heartbeats. As mentioned above, the monitoring system 100 can continuously acquire NIBP data (401) as well as ECG data (402) from the patient. The NIBP data can be filtered and sampled using a variety of techniques known in the art. For example, the system can include low frequency filters and/or high frequency filters to process the signals acquired to limit artifacts caused, for example, by movement or other artifact producing events.

Still with respect to FIG. 4, the acquired NIBP data and ECG data can be provided to a system algorithm. The system algorithm can detect NIBP oscillation pulses (405) and ECG waveforms (406). The system algorithm can classify the ECG waveforms (407) by comparing the detected waveform or heartbeat to a reference waveform or heartbeat. For example, the newly detected heartbeat can be compared to one or more heartbeat templates representing normal and abnormal heartbeats. The heartbeat templates can be dynamically updated beat by beat, for example during a relearn stage of the system. The heartbeat templates can allow the system to distinguish newly detected heartbeats that are normal from abnormal ones. One or more master templates for a particular patient can be generated in real-time and continually updated as additional data is retrieved from the patient. The master template can be what is considered to be the normal ECG waveform for that particular patient and can be made up of a number of heartbeats averaged. If a heartbeat does not closely resemble the normal averaged template, it can be discarded and the average recomputed. If the newly detected heartbeat does not match the template, the heartbeat can be identified by the system as abnormal. It should be appreciated that the master template can also include abnormal ECG waveforms for the patient and based on how well a newly detected heartbeat matches the abnormal template, the heartbeat can be identified by the system as normal or abnormal. The number of heartbeats collected and incorporated into the calculation for generating the reference or template can vary. It should be appreciated that the correlation process can vary and the above is provided for example only.

If the ECG waveform is classified as not normal (408), the system can reject (409) the ECG waveform and the detected NIBP pulse will not be saved to the pressure pulse profile. If the ECG waveform is classified as normal (408), the system can align the NIBP pulse to the ECG waveform (410) corresponding to a specific heartbeat. The pulse matching (411) phase can then be initiated using the aligned NIBP pulse.

It should be appreciated that the system algorithm can include one or more algorithms. For example, the monitoring system 100 can include one algorithm for analyzing the ECG data acquired and a separate algorithm for analyzing the NIBP data acquired. If both, the ECG and the NIBP algorithms are run on the same hardware, there can be a fixed system delay between the two algorithms on the monitoring system, for example, due to processing time and delay due to filtering and sampling rates. The fixed system delay can be characterized and determined in a lab experiment by collecting data for both signals and by using some software tools on a PC such as through a black-box system study. The time delay can be used to correlate and align each ECG-detected heartbeat with its corresponding NIBP pulse in real-time while the measurement is in progress. For example, after characterizing the system time delay with the ECG algorithm, the NIBP algorithm can use the beat class information as an input to examine the type of the beat and assess whether the ECG waveform corresponding to the heartbeat is normal or abnormal (e.g. PVC, PAC, etc.). If the ECG waveform corresponding to the heartbeat is normal, the NIBP algorithm can align the NIBP pulse and the ECG waveform to the corresponding heartbeat and include the NIBP pulse in the profile of the NIBP measurement. If the ECG waveform corresponding to the heartbeat is not normal, the NIBP pulse can be rejected and removed from the database so that it is not saved to the oscillation pulse profile. The rejection can happen in real-time as soon as ECG algorithm reports the most recent beat classification result.

Again with respect to FIG. 4, once the alignment of the NIBP pulse with the normal ECG waveform is performed, the pulse matching and trend analysis phase can be initiated. The pulse database at this stage can be devoid of any pulses aligning with abnormal heartbeats such that only pulses aligned to normal heartbeats are used during pulse matching (411). The NIBP pulses can be compared to neighboring NIBP pulses on a single step deflation or across steps. Similarity between NIBP pulses can be determined, for example, by assessing area, rise time, pulse amplitude, cross correlation and the like. NIBP pulses that are qualified (412) are stored to the pulse profile (413) for further use by the algorithm. NIBP pulses that are not qualified (412) are rejected (409).

The system algorithm can determine when a pressure pulse profile is complete (414). If the system algorithm determines the pressure pulse profile is complete, the profile will be curve fit (415) and systolic, diastolic, and MAP calculated (416). If the system algorithm detects the pressure pulse profile to be incomplete, reinflation of the cuff can be performed in order to acquire additional NIBP data (401) to fill in the missing data sections.

The algorithm can allow for at least 1, 2, 3, 4 or more reinflations to complete the pulse profile. Total measurement time can vary. A predefined maximum time period can be selected such that the measurement is complete or is stopped once the maximum time period is reached. In some implementations, total measurement time can be up to 100, 120, 140, 160, 180, 200, 220, 240, or more seconds for adults, up to 60, 80, 100, 120, 140, 160, 180 or more seconds for pediatrics, and up to 30, 45, 60, 75, 90, 100, 110 or more seconds for neonates. An additional timer limit can also be incorporated, for example, after the additional timer limit has been reached the cuff can be deflated completely and an algorithm retry initiated. The cuff can be inflated during a retry to 15 mm Hg above the last oscillation pulse detected in the profile. If no pulse detected in the profile, the cuff can be inflated to 160 mm Hg to perform the retry.

Various aspects of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, the memory, at least one input device, and at least one output device such as a display.

These computer programs (also known as programs, software, software applications or code) include machine instructions for a programmable processor, and may be implemented in a high-level procedural and/or object-oriented programming language, and/or in assembly/machine language. As used herein, the term “machine-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The implementations set forth in the foregoing description do not represent all implementations consistent with the subject matter described herein. Instead, they are merely some examples consistent with aspects related to the described subject matter. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows and steps for use described herein do not require the particular order shown, or sequential order, to achieve desirable results. Other embodiments can be within the scope of the claims.

1. A method for implementation by one or more data processors, comprising:
acquiring, by at least one data processor, noninvasive blood pressure data using an inflatable cuff applied around a portion of a patient by inflating the cuff to a pressure above the patient's systolic blood pressure, and detecting a first oscillation pulse within the cuff during a deflation step;
acquiring, by at least one data processor, electrocardiogram (ECG) data from the patient by detecting an ECG waveform corresponding to a first heartbeat using an ECG lead coupled to the patient;
classifying, by at least one data processor, the ECG waveform as normal or not normal;
aligning, by at least one data processor, the first oscillation pulse to the ECG waveform corresponding to the first heartbeat if the ECG waveform is classified normal or rejecting the first oscillation pulse if the ECG waveform is classified not normal; and
calculating, by at least one data processor, the blood pressure using the aligned first oscillation pulse.

2. The method of claim 1, further comprising:
detecting, by at least one data processor, a second oscillation pulse within the cuff during a deflation step, wherein the second oscillation pulse neighbors the first oscillation pulse;
acquiring, by at least one data processor, electrocardiogram (ECG) data from the patient by detecting a second ECG waveform characterizing a second heartbeat using the ECG lead coupled to the patient;
classifying, by at least one data processor, the second ECG waveform as normal or not normal;
aligning, by at least one data processor, the second oscillation pulse to the second ECG waveform corresponding to the second heartbeat if the second ECG waveform is classified normal or rejecting the second oscillation pulse if the second ECG waveform is classified not normal; and
performing, by at least one data processor, oscillation pulse matching between the aligned first oscillation pulse and the aligned second oscillation pulse to identify whether the first oscillation pulse qualifies for inclusion in calculating the blood pressure calculation.

3. The method of claim 2, wherein the second oscillation pulse occurs within the same deflation step as the first oscillation pulse.

4. The method of claim 2, wherein the second oscillation pulse occurs within a different deflation step as the first oscillation pulse.

5. The method of claim 2, wherein pulse matching comprises identifying similarity between at least one of area, rise time, and pulse amplitude of the first and second oscillation pulses.

6. The method of claim 2, wherein the first oscillation pulse qualified for inclusion in calculating the blood pressure calculation is stored to an oscillation pulse profile.

7. The method of claim 6, wherein the oscillation pulse profile comprises additional qualified oscillation pulses detected within the cuff.

8. The method of claim 6, further comprising assessing, by at least one data processor, whether the oscillation pulse profile is complete.

9. The method of claim 8, further comprising fitting, by at least one data processor, the completed oscillation pulse profile to a curve.

10. The method of claim 9, wherein calculating the blood pressure comprises calculating systolic, diastolic and mean arterial blood pressure of the patient from the oscillation pulse profile fit to the curve.

11. The method of claim 1, wherein calculating the blood pressure comprises calculating systolic, diastolic and mean arterial blood pressures of the patient.

12. The method of claim 1, wherein the ECG waveform classified as not normal is selected from the group consisting of premature ventricular contraction (PVC), premature atrial contractions (PAC), atrial fibrillation, atrial flutter, paroxysmal supraventricular tachycardia (PSVT), accessory pathway tachycardias, AV nodal reentrant tachycardia, ventricular tachycardia (V-tach), ventricular fibrillation, long QT syndrome, bradyarrhythmias, sinus node dysfunction, and heart block.

13. A system comprising:
a noninvasive blood pressure monitor to provide noninvasive blood pressure data;
an electrocardiogram sensor to provide electrocardiogram data;
at least one data processor;
memory, coupled to the at least one data processor, storing instructions, which when executed, cause the at least one data processor to perform operations comprising:
acquiring noninvasive blood pressure data using the noninvasive blood pressure monitor, and detecting a first oscillation pulse;
acquiring electrocardiogram (ECG) data from the electrocardiogram sensor by detecting an ECG waveform corresponding to a first heartbeat using an ECG lead;
classifying the ECG waveform as normal or not normal;
aligning the first oscillation pulse to the ECG waveform corresponding to the first heartbeat if the ECG waveform is classified normal or rejecting the first oscillation pulse if the ECG waveform is classified not normal; and
calculating the blood pressure using the aligned first oscillation pulse.

14. A non-transitory computer program product storing instructions, which when executed by at least one data processor of at least one computing system, implement operations comprising:
acquiring noninvasive blood pressure data using an inflatable cuff a lied around a portion of a patient by inflating the cuff to a pressure above the patient's systolic blood pressure and detecting a first oscillation pulse within the cuff during a deflation step;
acquiring electrocardiogram (ECG) data from the patient by detecting an ECG waveform corresponding to a first heartbeat using an ECG lead coupled to the patient;
classifying the ECG waveform as normal or not normal;
aligning the first oscillation pulse to the ECG waveform corresponding to the first heartbeat if the ECG waveform is classified normal or rejecting the first oscillation pulse if the ECG waveform is classified not normal; and
calculating the blood pressure using the aligned first oscillation pulse.

15. The non-transitory computer program product of claim 14, the operations further comprising:
detecting, by at least one data processor, a second oscillation pulse within the cuff during a deflation step, wherein the second oscillation pulse neighbors the first oscillation pulse;
acquiring, by at least one data processor, electrocardiogram (ECG) data from the patient by detecting a second ECG waveform characterizing a second heartbeat using the ECG lead coupled to the patient;
classifying, by at least one data processor, the second ECG waveform as normal or not normal;
aligning, by at least one data processor, the second oscillation pulse to the second ECG waveform corresponding to the second heartbeat if the second ECG waveform is classified normal or rejecting the second oscillation pulse if the second ECG waveform is classified not normal; and
performing, by at least one data processor, oscillation pulse matching between the aligned first oscillation pulse and the aligned second oscillation pulse to identify whether the first oscillation pulse qualifies for inclusion in calculating the blood pressure calculation.

16. The non-transitory computer program product of claim 15, wherein the second oscillation pulse occurs within the same deflation step as the first oscillation pulse.

17. The non-transitory computer program product of claim 15, wherein the second oscillation pulse occurs within a different deflation step as the first oscillation pulse.

18. The non-transitory computer program product of claim 15, wherein pulse matching comprises identifying similarity between at least one of area, rise time, and pulse amplitude of the first and second oscillation pulses.

19. The non-transitory computer program product of claim 15, wherein the first oscillation pulse qualified for inclusion in calculating the blood pressure calculation is stored to an oscillation pulse profile.

20. The non-transitory computer program product of claim 19, wherein the oscillation pulse profile comprises additional qualified oscillation pulses detected within the cuff.
